(19) United States
(12) Plant Patent Application Publication
(10) Pub. No.: US 2002/0104146 P1
(43) Pub. Date: Aug. 1, 2002

(54) GERANIUM PLANT NAMED 'FISRORED'

(75) Inventor: Angelika Utecht, Montabaur (DE)

Correspondence Address: Foley & Lardner, Suite 500, 3000 K Street, N.W., P.O. Box 25696, Washington, DC, 20007-8696, US

(73) Assignee: FLORFIS AG

(21) Appl. No.: 09/773,003

(22) Filed: Jan. 31, 2001

(51) Int. Cl. A01H 5/00

(52) U.S. Cl. PLT/330

(57) ABSTRACT

A new and distinct cultivar of geranium plant named ‘Fisrored’, as described and illustrated, and particularly characterized by the combined features of deep red, semi-double flowers, large, many-flowered umbels, foliage with strong zonation, vigorous growth and moderately tall plant habit, and medium (mid season) begin of flowering.

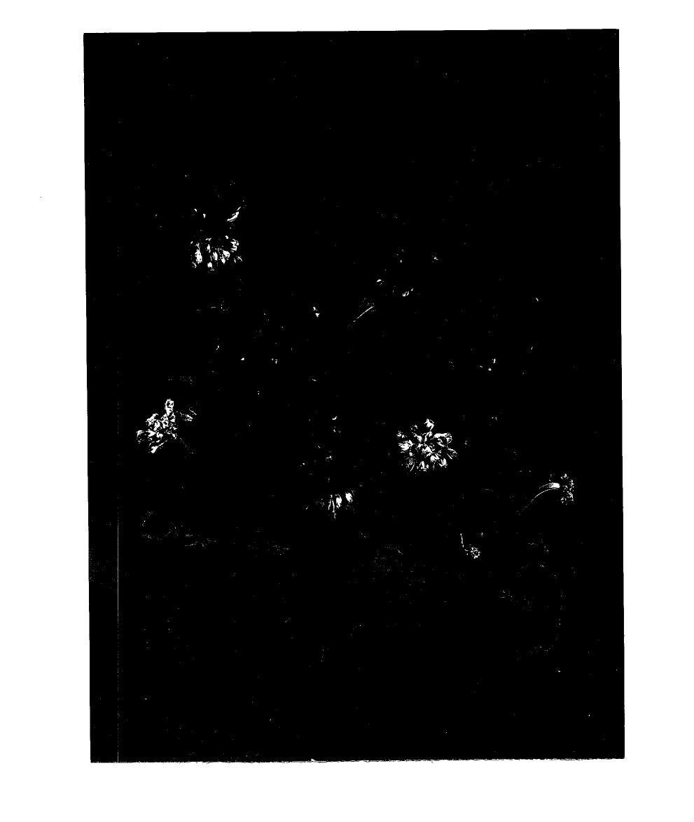

### BACKGROUND OF THE INVENTION

The present invention comprises a new and distinct cultivar of geranium, botanically known as Pelargonium zonale, and hereinafter referred to by the cultivar name ‘Fisrored’.

‘Fisrored’ is a product of a planned breeding program which had the objective of creating new geranium varieties with deep red flower color.

‘Fisrored’ was originated from a hybridization made by the inventor Angelika Utecht in a controlled breeding program in Galdar, Gran Canaria, Spain, in 1994.

The female parent was a hybrid seedling, no. 1477-2, having intense red, single-type flowers, large inflorescence, large, dark-green leaves and medium plant habit. The male parent of ‘Fisrored’ was the variety ‘Fistador’, U.S. plant patent applied for Ser. No. 09/323,149, which is characterized by scarlet semi-double flowers, zoned foliage, and medium-tall, round plant habit.

‘Fisrored’ was selected as one flowering plant within the progeny of the stated cross by Angelika Utecht in 1995 in a controlled environment in Galdar, Gran Canaria, Spain. The first act of asexual reproduction of ‘Fisrored’ was accomplished when vegetative cuttings were taken from the initial selection in the fall of 1995 in a controlled environment in Galdar, Gran Canaria, Spain, by, or under the supervision of, Angelika Utecht.

Horticultural examination of plants grown from cuttings of the plant initiated in May 1997 in Hillscheid, Federal Republic of Germany, and continuing thereafter, has demonstrated that the combination of characteristics as herein disclosed for ‘Fisrored’ are firmly fixed and are retained through successive generations of asexual reproduction.

‘Fisrored’ has not been observed under all possible environmental conditions. The phenotype may vary significantly with variations in environment such as temperature, light intensity and day length.

### BRIEF DESCRIPTION OF THE INVENTION

The following observations, measurements, and comparisons describe plants grown in Hillscheid, Germany, and in Langley, British Columbia, Canada, under greenhouse conditions which approximate those generally used in commercial practice.

The following traits have been repeatedly observed and are determined to be basic characteristics of ‘Fisrored’ in combination distinguish this geranium as a new and distinct cultivar

1. deep red (true red) flower color, semi-double, round florets;

2. large inflorescence with many flowers, tightly arranged;

3. medium-green foliage with strong zonation;

4. vigorous growth, and relatively tall, bushy plant habit; and

5. mid season spring flowering response.

Of the many commercial cultivars known to the present inventor, the most similar in comparison to ‘Fisrored’ is the parental variety ‘Fistador’. In comparison to ‘Fistador’, ‘Fisrored’ has a similar flower color, longer peduncles and larger inflorescence, and considerably taller plant habit, making it more suitable for extensive outdoor planting.

### BRIEF DESCRIPTION OF THE DRAWING

The accompanying photographic drawing shows typical flower and foliage characteristics of ‘Fisrored’ with colors being as true as possible will illustrations of this type.

The measurements were taken in Langley, British Columbia, Canada, on Jul. 20, 2000, 15 weeks after planting of rooted cuttings into 6 inch pots. The plants had not been pinched.

In the following description color references are made to The Royal Horticultural Society Colour Chart (RHS). The color values were determined indoors from flowers developed in a green-house in May, 2000, in Hillscheid, Germany.

Classification:

Botanical.—A hybrid of the species Pelargonium zonale L'Herit.

Commercial.—Zonal geranium, cv. ‘Fisrored’.

Inflorescence:

Umbel.—Shape: Flat and wide, nearly semi-spherical Average diameter: 117 mm Peduncle length: 148 mm Peduncle color: Fresh-green, RHS 143 A Pedicel length: 34 mm Pedicel color: Reddish to dark red (upper part) RHS 181 A to RHS 60 A Number of flowers per umbel: About 40.

Corolla.—Average diameter: 43 mm Form: Semi-double Shape: Small to medium-sized, round, cup-shaped Number of petals: 7-9 Number of petaloids: 1-2 Color (general tonality from a distance of three meters): Intense medium red Color of upper petals: Between RHS 43 A and 45 B Markings of upper petals: Weak dark-red veins Color of lower petals: Between RHS 43 and 45 B, no markings Color of lower surface of petals: RHS 43 A-B Color of sepals: Green, base dark-red, RHS 60 A Number of sepals: 5.

Bud.—(just prior to petals unfolding) Shape: Elliptical Color (lower part—sepals): Mainly green, RHS 137 D, some infusion of anthocyanin at the base Color (upper part—petals) Orange-red, RHS 43 A Length: 15 mm Width: 9 mm.

Reproductive organs.—Androecium: About 5 fertile anthers, whitish to pink filaments, yellow-orange pollen Gynoecium: 5-6-lobed stigma,, whitish filament Fertility/seed set: Occasionally a few seeds are developed.

Spring flowering response period.—In Hillscheid, Germany, in 2000, plants had on average 0.8 flowers opened 8 weeks after planting of rooted cuttings

Outdoor flower production.—Medium number of inflorescence, the flower count in 2000, in Hillscheid, Germany, indicated about 3-4 inflorescence per plant in mid-May.

Durability.—Good stability of flower color, relatively good rain resistance

Plant:

Foliage.—Form: Kidney-shaped, with open base Margin: Bicrenated Size of leaf: 97 mm wide Color of upper surface: Medium- green, approximately RHS 137 B Color of zonation: Strong, brown, about RHS 166 A Tolerance of botrytis: Typical.

General appearance and form.—Stem color: Green, RHS 143 A Internode length: 10-20 mm Branching pattern: 5.8 branches Height of foliage: 23.5 cm/36.5 cm wide.

I claim:

1. A new and distinct cultivar of geranium plant named ‘Fisrored’, as described and illustrated.
